(19) United States
(12) Patent Application Publication
(10) Pub. No.: US 2007/0021710 A1
(43) Pub. Date: Jan. 25, 2007

(54) Tampon applicator having a multi-directional rim

(76) Inventors: Eugenia Maren Bertulis, Cincinnati, OH (US); Michael Edward Carrier, Monfort Heights, OH (US); Diane Lee Norris, Wyoming, OH (US); Michael Sean Farrell, Terrace Park, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/187,173

(22) Filed: Jul. 22, 2005

(51) Int. Cl. A61F 13/20

(52) U.S. Cl. 604/15

(57) ABSTRACT

A tampon applicator having an outer member and an inner member. The outer member has an insertion end and an inner member receiving end. The inner member receiving end has a multi-directional rim. The inner member has a first end and a second end. The second end has a multi-directional rim.

### FIELD OF THE INVENTION

The present invention relates to an insertion device capable of housing an insertable element. More particularly, the present invention relates to a catamenial insertion device, such as a tampon applicator, having a multi-directional rim.

### BACKGROUND OF THE INVENTION

Applicators for delivering materials into a body cavity typically comprise a tubular insertion member having an insertion end and a gripper end opposite thereof, and an elongate expulsion member slideably fitted within the tubular insertion member for expelling the contained materials. The gripper end will generally incorporate features to allow a user to more or less securely hold the applicator during use—inserting the applicator into a body cavity, expelling a substantially enclosed material contained by the applicator, and withdrawing the applicator from the body.

Attempts have been made to improve the user's ability to manipulate the applicator during use. One approach is to significantly reduce the diameter of the applicator in the gripper end. Another approach is to provide a finger gripping portion of a tube with a series of slits, and then compressing the gripping portion to form a shoulder at a singular point of diameter change. While the reduced diameter grip of these applicators may help in preventing fingers from slipping towards the insertion end during the insertion step, there is little or no resistance offered in the opposite direction during the expulsion step. This is a step with which many users have difficulty.

In view of the shortcomings of the prior art, what is needed is an applicator which has substantial resistance to finger slip during both applicator insertion into a body cavity and expulsion of material contained by the applicator.

While addressing the need to assist the user with an applicator for insertion of a pledget into the vagina, the prior art gives little weight to the need to be configured to also provide equal ability upon removal of the applicator outer member from the vagina. Thus, there is a need for a tampon applicator with a distinct multi-directional rim area having at least one multi-directional structure that allows a consumer to use the multi-directional rim of the applicator for insertion of the applicator and expulsion of the pledget with ease, but also use the multi-directional rim of the applicator for easy removal from the vagina.

### SUMMARY OF THE INVENTION

The present invention encompasses a tampon applicator having an outer member and an inner member. The outer member has an insertion end and an inner member receiving end. The inner member receiving end has a multi-directional rim. The inner member has a first end and a second end. The second end has a multi-directional rim.

### BRIEF DESCRIPTION OF THE DRAWINGS

FIG. 1A is a perspective view of the tampon applicator of the present invention.

FIG. 1B is a perspective view of an alternative embodiment of the tampon applicator of the present invention.

FIG. 1C is a perspective view of an alternative embodiment of the tampon applicator of the present invention

FIG. 2 is a perspective view of the inner member of the tampon applicator.

FIG. 3A is a perspective view of an alternative embodiment of the tampon applicator of the present invention

FIG. 3B is a perspective view of the outer member of the tampon applicator.

### DETAILED DESCRIPTION OF THE INVENTION

While the present invention generally relates to devices having a tubular member with a multi-directional rim disposed at one end thereof, the following detailed description will refer, specifically, to a tampon applicator, a particularly preferred embodiment. One of ordinary skill in the art will recognize other uses for this invention including, without limitation, applicators for other catamenial devices, such as collection cups, inflatable plugs or cups, and the like; incontinence devices; suppositories; nasal packing; and the like.

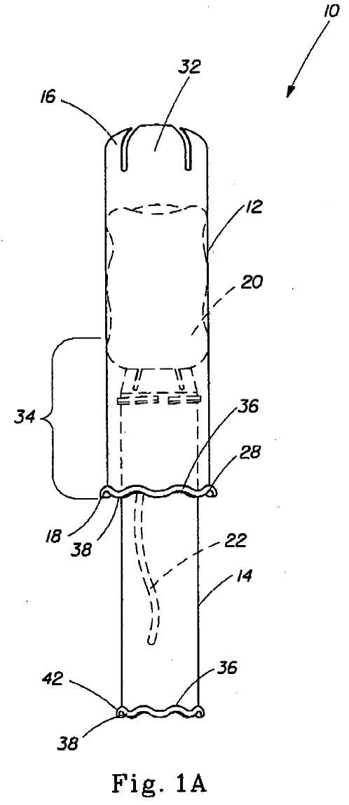

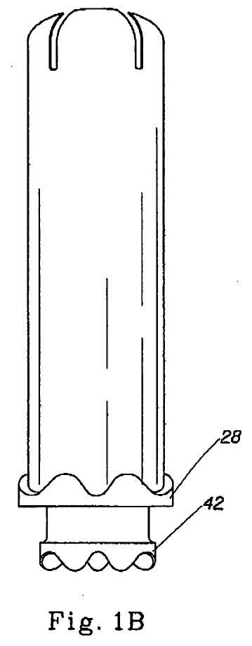

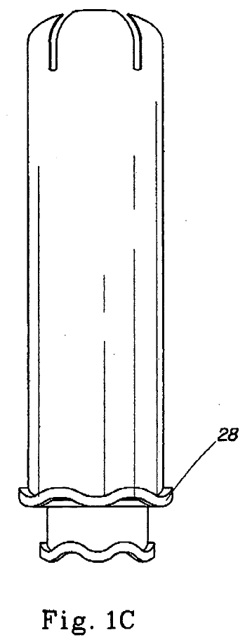

FIG. 1A discloses a tampon applicator 10 according to the present invention formed of an outer member 12 and an inner member 14.

The outer member 12 has an insertion end 16 and an inner member receiving end 18, and it is arranged and configured to contain a tampon 20 having a withdrawal string 22. The inner member receiving end 18 comprises a multi-directional rim 28.

The insertion end 16 may be open-ended or may have three to eight separate or connected petals 32, which provide increased comfort to the user during insertion and removal of applicator 10. When the insertion end 16 of the applicator 10 has petals 32, the petals 32 enclose the tampon 20 during insertion of applicator 10 into the vagina, resulting in increased comfort to the user.

To enhance gripping of outer member 12, a fingergrip area 34 may be disposed on or in the outside surface of outer member 12. In one embodiment, fingergrip area 34 is disposed about the circumference or outer surface of outer member 12. More preferably, fingergrip area 34 is disposed adjacent the multi-directional rim 28.

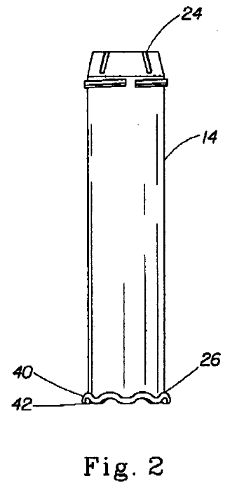

Referring to FIG. 2, the inner member 14 has a first end 24 and a second end 26 generally directed toward the insertion end 16 (FIG. 1A) and inner member receiving end 18 (FIG. 1A) of the outer member 12 (FIG. 1A), respectively. The first end 24 of the inner member 14 is arranged and configured to be slideably introduced into the inner member receiving end 18 of the outer member 12 (FIG. 1A).

The second end 26 of the inner member 12 may also comprise at least one multi-directional rim 40. In an alternative embodiment of the present invention illustrated in FIG. 2, the inner member 14 may have a multi-directional rim 40 disposed at the edge 42 associated with the second end 26.

Referring to FIG. 1A, the tampon applicators 10 or other tubular devices of the present invention can have tube geometries or cross-sections that are useful to contain the object to be inserted. Often, the shape of the tampon 20 contained suggests the shape of the outer member 12, but departures from this general rule may be made. Therefore, the outer member 12 may take on numerous cross-sectional shapes including, without limitation, circular, oval, polygonal (e.g., trapezoidal, rectangular, triangular), and the like. For example, cylindrical tampons 20 may be contained within rectangular outer members 12 and trapezoidal tampons 20 (such as those disclosed in Van Iten et al., U.S. Pat. No. 5,350,371) and cup-shaped tampons 20 (such as those disclosed in Bailey, U.S. Pat. No. 2,330,257) can be contained in a generally cylindrical outer member 12. In addition, the outer member 12 can be substantially elongated, curved, or flexible, or it can take on other shapes that are apparent to one of ordinary skill in the art. The specific geometry, itself, is not critical to the practice of the present invention.

A. Multi-Directional Rim

The inner member receiving end 18 of the outer member 12 may comprise at least one multi-directional rim 28. Moreover, the second end 26 of the inner member 14 may comprise at least one multi-directional rim 42. The pattern of the multi-directional rim 28 for the outer member 12 and the pattern of the multi-directional rim 42 for the inner member 14 may be the same, substantially the same, or different. The patterns for the multi-directional rims 28, 42 are substantially the same if the patterns closely resemble each other or if one pattern has the possibility of being mistaken for the other. A pattern is the “same” if the pattern corresponds so closely that is it indistinguishable. Referring to FIG. 1A, the pattern for the multi-directional rims 28, 42 are the same. Referring to FIG. 1B, in another embodiment, the pattern/shape for the multi-directional rims 28, 42 are substantially the same.

The multi-directional rims 28,42 may be raised above the outer surface of outer member 12, inner member 14, depressed below the outer surface of outer member 12, inner member 14 constructed so as the tip of the multi-directional rims 28,42 aligns with the outer surface of outer member 12, or any combinations thereof. The multi-directional rims 28,42 may be patterned or arranged in any configuration and in any number suitable for creating an enhanced gripping area for a user's fingers and/or a greater surface area for a stop to assist during the removal of the applicator 10.

i. Multi-Directional Rim Regions

Tampons 20, and notably radially expanding tampons 20, are capable of exerting increased forces on applicator outer member 12, making it difficult for a tampon user to expel the tampon 20 into the vagina. Once the consumer has expelled tampon 20 into the vagina from outer member 12, it is equally important that the consumer have a suitable gripping ability on applicator 10 to easily overcome removal forces exerted on the applicator 10 by the vaginal walls and remove the applicator 10 from the vagina.

In summary, a woman can securely and comfortably grasp, control and position the tampon applicator 10 of the present invention, during insertion of applicator 10, expulsion of tampon 20 housed therein, and subsequent removal of the applicator from the vagina, as a result of a multi-directional rim 28, 40. Moreover, the multi-directional rims 28, 40 provide an increased surface area which creates better control of the tampon applicator 10 during removal of the tampon applicator 10. In addition, the multi-directional rim 28, 40 structure provides gripping ability axially in both the insertion and removal directions without the user having to change their grip on outer member 12, so the user is able to easily maneuver, control and position the applicator.

Each multi-directional rim 28, 42 encompasses a first region 36 and a second region 38. The multi-directional rims 28,42 are arranged substantially in columns or rows extending axially from the inner member receiving end 28 of the outer member 12. To provide multi-directional contact by the user, a first region 36 of the multi-directional rims 28,42 is disposed in a first, insertion direction in one or more columns and in a second region 38 of the multi-directional rims 28,42, removal direction, are substantially opposite the first direction, in one or more remaining columns. As a result, a tampon user naturally contacts their finger(s) with the multi-directional rims 28,42, due to the increased surface area of the multi-directional rims 28,42, disposed in the same direction as the applicator, i.e., insertion direction during insertion of the applicator and removal direction during removal of the applicator 10.

When a user's fingers contacts the multi-directional rims 28,42 in both the insertion and removal directions, the user not only experiences enhanced grippability in the direction of travel of the applicator as a result of those multi-directional rims 28,42 disposed in that same direction, the gripping structures disposed in the opposite direction also provide additional friction, which also enhances grippability of the applicator 10. As a result, the multi-directional gripping structures ensure that a user can insert an applicator, eject a tampon 20 from the applicator, and remove an applicator with comfort and ease.

The regions 36, 38 of the multi-directional rims 28, 42 may encompass the entire perimeter or may encompass part of the perimeter of the outer member 12, inner member 14. In one non-limiting example, the regions 36, 38 of the multi-directional rims 28, 42 are spaced discontinuously around the perimeter of the inner member receiving end 28, second end 26.

ii. Number, Shape, and Size of the Multi-Directional Rim Regions

The regions 36, 38 of the multi-directional rims 28, 42 can be of any number, shape, color, or size as long as the regions 36, 38 of the multi-directional rims 28, 42 are able to create a structure which contacts the user. Any number of regions 36, 38 of the multi-directional rims 28, 42 may be utilized. The regions 36, 38 can be any number as long as the regions 36, 38 do not resemble a substantially straight line. In one non-limiting example, the multi-directional rim 42 can have about 100 regions 36 and about 100 regions 38. The regions 36, 38 can be any shape as long as such shape would work effectively to contact the user during insertion of the tampon applicator 10 and during withdrawal of tampon applicator 10. While regions 36, 38 are shown, these could be replaced by raised areas of any desired shape. The regions 36, 38 may be any three-dimensional geometric shape known including but not limited to arcs, ovals, circles, rectangles, trapezoids, triangles, cones, alphabet letters, and mixtures thereof. Moreover, regions 36, 38 features may take the form of bumps, pyramids, rings, recesses, holes, and the like. In one non-limiting example, the regions 36, 38 may form the pattern of two rows. In another non-limiting example, referring to FIG. 1C, another embodiment of the multi-directional rim 28 is shown.

In one embodiment of the present invention, as depicted in FIG. 1A, the multi-directional rims 28,42 are two or more regions 36,38 formed in an arcuate configuration. With this configuration, arcuate regions 36 is in a first, insertion direction and arcuate regions 38 is in a second, substantially opposite second direction in adjacent columns, thereby providing multi-directional rims 28,42. By alternating the direction of the regions 36,38 in each column, the edge or shoulder is formed in an opposite direction in each column, thus providing effective contact with the multi-directional rims 28,42 and gripping in both the insertion direction and removal direction of applicator 10.

The plurality of regions can be straight regions or slits 36,38 as long as each region 36,38 is in substantially opposite directions. Each straight region 36,38 is formed such that each region 36,38 is substantially straight across. However, regions 36,38 are in substantially opposite directions. Each region 36,38 can be raised or depressed relative to the outer surface of outer member 12 upon formation. By alternating the direction of the straight region 36,38 in each column, region 36,38 is formed in an opposite direction in each column, thus providing effective gripping in both the insertion direction and removal direction of applicator 10. It should be understood that the straight region 36,38 can be arranged or patterned in any configuration and in any number suitable for creating an enhanced gripping area, as set forth above for the arcuate region 36,38 embodiment.

Also, it should be understood that a tampon applicator can be formed with a multi-directional rims 28,42 having any combination of straight regions and arcuate regions. These regions can be arranged or patterned in any configuration and in any number suitable for creating an enhanced gripping area.

While FIG. 1A depicts each column with regions 36,38 disposed in alternating axial directions, i.e., insertion and removal directions, the columns may be arranged such that two or more adjacent columns have multi-directional rims 28,42 disposed in the same direction with two or more subsequent adjacent columns having multi-directional rims 28,42 disposed in a substantially opposite direction. It should be understood that the columns of multi-directional rims 28,42 can be arranged in any pattern such that a suitable number of multi-directional rims 28,42 are disposed in opposite directions to one another to ensure adequate axial gripping ability in both the insertion and removal directions.

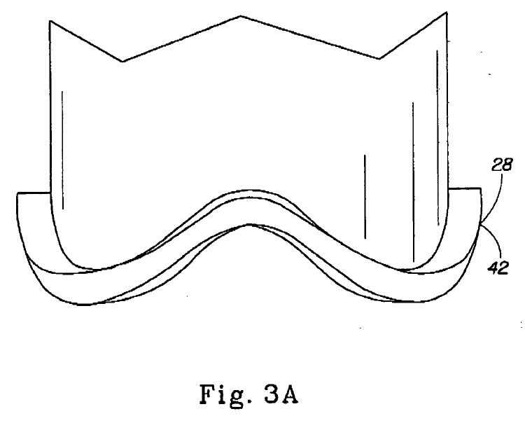

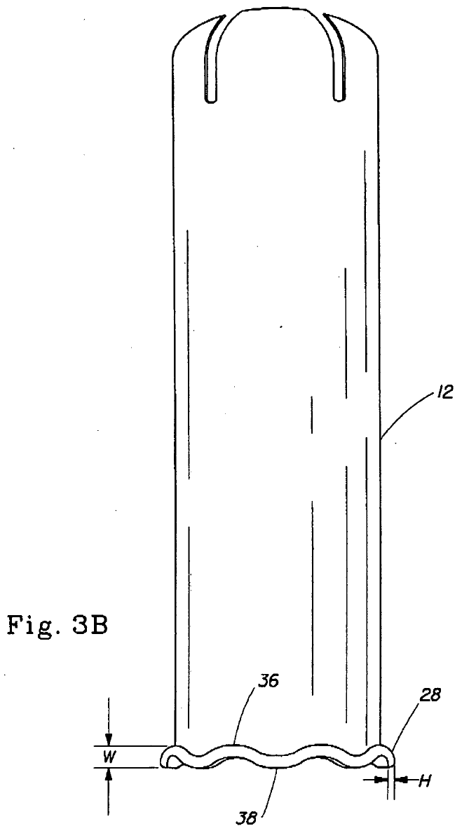

It should be understood that the multi-directional rims 28,42 may be arranged in any number of columns practical, given the circumference of the applicator outer member and/or fingergrip area 34. It should also be understood that each multi-directional rims 28,42 in any given substantial column may vary slightly in size, dimension and/or shape from other gripping structures in that same column. In one embodiment, the multi-directional rims 28,42 have two or more multi-directional rims 28,42 arranged in about 4 to about 8 columns. When a smaller number of columns is used, i.e., about 4, the columns may be arranged such that diametrically opposed columns have multi-directional rims 28,42 arranged in the same direction. Referring to FIG. 3A, in one non-limiting embodiment, two columns are shown for either the multi-directional rim 28 and/or multi-directional rim 42. Such an arrangement naturally urges a user's fingers to contact the multi-directional rims 28,42 disposed in the intended direction of the applicator. This results in greater ease of use of the applicator to the consumer.

Referring to FIG. 3B, the regions 36, 38 of the multi-directional rims 28, 42 may be any size. The size of the regions 36, 38 can increase by texture and/or material. Each region 36,38 preferably has a width dimension W between about 0.2 mms to about 63 mms. More preferably, each region 36,38 has a width dimension W between about 3.81 mms to about 6.35 mms. In addition, each region 36,38 can form a raised edge or shoulder having a height dimension H of about 0.254 mms to about 1.524 mms, and more preferably between about 0.508 mms to about 0.762 mms. The height dimension H is measured above or below the plane of the outer member 12. In other words, the height dimension H can be measured from the outer surface of the outer member 12 to the highest point of the region 36,38. In another embodiment, the height dimension H of the multi-directional rim 28, 40 (see FIG. 2 for reference number 40) can project outwardly from the inner/outer member 12,14 (see FIG. 2 for reference number 14) by at least about 0.1 mm, and more preferably, by at least about 1 mm, and most preferably, by at least about 10 mm. This outward projection is useful to provide a comfortable, finished edge to at least one member of an applicator, such as a tampon applicator.

Referring to FIG. 1A, the multi-directional rim 28 can have a maximum outside diameter that is greater than the minimum inner diameter of the outer member 12. Likewise, the multi-directional rim 42 can have a maximum outside diameter that is greater than the minimum inner diameter of the inner member 14. Alternatively, in a pair of tubular members in which the outer member 12 is cylindrical and the outer member 12 has a polygonal cross-section, the multi-directional rim 28 of the outer member 12 should have a maximum outside diameter that is greater than the minimum inner dimension of the outer member 12 passing through a central axis thereof. Alternatively, in a pair of tubular members in which the inner member 14 is cylindrical and the inner member 14 has a polygonal cross-section, the multi-directional rim 42 of the inner member 14 should have a maximum outside diameter that is greater than the minimum inner dimension of the inner member 14 passing through a central axis thereof.

While the specific tube geometry is not critical, in order for the multi-directional rim 42 to provide a stop, it must create an interference with at least part of the outer member 12. The multi-directional rim 42 and/or the length of inner member 14 can provide a stop to allow a user to determine when the contents of the tampon applicator 10 have been fully expelled. This feature also prevents the inner member 14 from being pushed or pulled through the outer member 12 and being left behind in the body cavity. This interference can be accomplished in any numbers of ways. Preferably, the interference is accomplished by configuring the multi-directional rim 42 such that at least a portion of the multi-directional rim 42 must project beyond an area defined by the inner dimensions of the outer member 12. For example, in a pair of cylindrical tubular members, the multi-directional rim 42 of the inner member 14 should have a maximum outer diameter that is greater than the minimum inside diameter of the outer member 12. Alternatively, in a pair of tubular members in which the inner member 14 is cylindrical and the outer member 12 has a polygonal cross-section, the multi-directional rim 42 of the inner member 14 should have a maximum outside diameter that is greater than the minimum inner dimension of the outer member 12 passing through a central axis thereof. In yet another alternative, in which the inner member 14 has a rectangular cross-section and the outer member 12 is cylindrical, the maximum length of a side of the multi-directional rim 42 is greater than a corresponding chord of the circle defined by the inner diameter of the outer member 12. The term “diameter” as used herein the specification and the claims means a chord passing through the center of a figure or body; the length of a straight line through the center of an object.

iii. Joining of Multi-directional Rim

The multi-directional rims 28, 40 can be joined to the outer member 12, inner member 14 or formed in the outer member 12, inner member 14 in any way known in the art. The multi-directional rims 28, 40 can also be monolithic with the outer member 12, inner member 14, for example, by molding the multi-directional rims 28, 40 and the outer member 12, inner member 14 as one piece. In one non-limiting example, the multi-directional rims 28, 40 can be joined to the outer member 12, inner member 14 by friction fitting that snaps parts together, gluing, and/or melting.

B. Tampon Applicator Materials

The applicator devices of the present invention can be made of materials generally known to those of ordinary skill in the art. The applicators may be plastic, such as injection-moldable or blow-moldable plastic, biodegradable plastic, such as those disclosed in Dabi et al., U.S. Pat. No. 5,910,520 (herein incorporated by reference), or cardboard. The cardboard used in tampon applicators can be a single layer of cardboard material, or it can be a plurality of laminated layers to provide multiple benefits relating to the various layers. Useful cardboard stock for the formation of the tubular elements include, without limitation, paperboard, cardboard, cup stock, paper, and the like. The laminated cardboard material may include a surface layer or coating of plastic, wax, silicone, and the like, which may be useful to increase the comfort to the user during insertion and withdrawal. The plastic coating may include, without limitation, polyolefins such as polyethylene and polypropylene, polyesters such as polyethylene terephthalate, polycaprolactone, polyvinyl alcohol, ethylene-vinyl acetate copolymers, cellophane, and the like.

Preferred tubular element materials include laminated cardboards. Preferred laminated cardboards include plastic laminated or plastic coated cardboard materials. These plastic laminated cardboard materials may include additional layers such as adhesive layers, tie layers, and the like.

Typical dimensions for the tubular elements useful in tampon applicators include a length of about 5 to 8 cm, a diameter of about 8 to 20 mm, and thicknesses of about 0.1 to 0.6 mm. Preferably, the diameter of the inner tubular element is less than the diameter of the outer tubular element to allow for a telescopic arrangement of the inner tubular element within the outer tubular element as shown in FIG. 1A. The applicators can be made by the following non-limiting processes: spiral winding as disclosed in U.S. Pat. No. 5,346,468, convolute winding as disclosed in U.S. Pat. No. 4,508,531, and forming a sheet around a mandrel and then sealing an overlapped seam as disclosed in U.S. Pat. No. 4,755,164.

The foregoing specification and drawings are merely illustrative of the present invention and are not intended to limit the present invention to the disclosed embodiments. Variations and changes, which are obvious to one skilled in the art, are intended to be within the scope and nature of the present invention as defined in the appended claims.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A tampon applicator comprising:
A.) an outer member comprising an
i.) an insertion end
ii.) and an inner member receiving end, wherein said inner member receiving end comprises a multi-directional rim, wherein said multi-directional rim comprises
a.) an outer member first region and an outer member second region,
b.) wherein said outer member first region is directed in a first direction and said outer member second region is directed in a second direction, wherein said first direction is substantially opposite said second direction and
B.) an inner member comprising
i.) a first end and
ii.) a second end, wherein said second end comprises a multi-directional rim,
a.) said multi-directional rim comprises an inner member first region and an inner member second region,
b.) wherein said inner member first region is directed in a first direction and said inner member second region is directed in a second direction, wherein said first direction is substantially opposite said second direction.

2. The tampon applicator of claim 1, wherein said multi-directional rim of said outer member has said outer member first region comprising an outer member first shape and said outer member second region comprising an outer member second shape.

3. The tampon applicator of claim 2, wherein said outer member first shape of said outer member first region and said outer member second shape of said outer member second region have a same shape.

4. The tampon applicator of claim 2, wherein said outer member first shape of said outer member first region and said outer member second shape of said outer member second region have different shapes.

5. The tampon applicator of claim 2, wherein said outer member first shape of said outer member first region is selected from the group consisting of arcs, straight lines, ovals, circles, rectangles, trapezoids, triangles, cones, alphabet letters, and mixtures thereof.

6. The tampon applicator of claim 2, wherein outer member said second shape of said outer member second region is selected from the group consisting of arcs, ovals, circles, rectangles, trapezoids, triangles, cones, alphabet letters, and mixtures thereof.

7. The tampon applicator of claim 2, wherein said outer member first and outer member second regions are arranged in a pattern selected from the group consisting of waves, flowers, ovals, circles, rectangles, trapezoids, triangles, cones, alphabet letters, and mixtures thereof.

8. The tampon applicator of claim 2, wherein said multi-directional rim of said inner member has said inner member first region comprising an inner member first shape and said inner member second region comprising an inner member second shape.

9. The tampon applicator of claim 8, wherein said inner member first shape of said inner member first region and said inner member second shape of said inner member second region have a same shape.

10. The tampon applicator of claim 8, wherein said inner member first shape of said inner member first region and said inner member second shape of said inner member second region have different shapes.

11. The tampon applicator of claim 8, wherein said inner member first shape of said inner member first region is selected from the group consisting of arcs, straight lines, ovals, circles, rectangles, trapezoids, triangles, cones, alphabet letters, and mixtures thereof.

12. The tampon applicator of claim 8, wherein said inner member second shape of said inner member second region is selected from the group consisting of arcs, straight lines, ovals, circles, rectangles, trapezoids, triangles, cones, alphabet letters, and mixtures thereof.

13. The tampon applicator of claim 8, wherein said inner member first and inner member second regions are arranged in a pattern selected from the group consisting of waves, flowers, ovals, circles, rectangles, trapezoids, triangles, cones, alphabet letters, and mixtures thereof.

14. The tampon applicator of claim 1, wherein said insertion end of said outer member further comprises petals.

15. The tampon applicator of claim 1, wherein said multi-directional rim of said outer member and said multi-directional rim of said inner member have at least about two first regions and two second regions.

16. The tampon applicator of claim 1, wherein said tampon applicator is selected from the group consisting of plastic, wax, silicone, biopolymer, cellophane, nitrocellulose, lacquer, epoxy, and any combinations thereof.

17. The tampon applicator of claim 1, wherein said tampon applicator is selected from the group consisting of paper, paper laminate, paper-based, plastic, paper slurry, biopolymer, and any combinations thereof.

18. A tampon applicator comprising:
a.) an outer member comprising an
i. an insertion end and
ii. an inner member receiving, wherein said inner member receiving end comprises a multi-directional rim; and
b.) an inner member comprising an
i. a first end and
ii. a second end, wherein said second end comprises a multi-directional rim.
